(19) United States
(12) Patent Application Publication
(10) Pub. No.: US 2021/0121173 A1
(43) Pub. Date: Apr. 29, 2021

(54) DEVICE FOR SUTURING TISSUE TO CLOSE DEFECTS

(71) Applicant: The Board of Trustees of the Leland Stanford Junior University, Stanford, CA (US)

(72) Inventors: Eric Anthony Kramer, Los Altos Hills, CA (US); Vivian Emmely de Ruijter, Menlo Park, CA (US); Katherine Sherman Blevins, Mountain View, CA (US); Chirag Ambaram Chauhan, Bethel Park, PA (US)

(21) Appl. No.: 16/605,010

(22) PCT Filed: Apr. 19, 2018
(86) PCT No.: PCT/US2018/028296

§ 371 Date: Oct. 13, 2019

Related U.S. Application Data
(60) Provisional application No. 62/486,978, filed on Apr. 19, 2017.

(51) Int. Cl. A61B 17/04; A61B 17/062; A61B 17/00

(52) U.S. Cl. CPC A61B 17/0482; A61B 17/0625; A61B 17/0469; A61B 2017/00663; A61B 2017/0472; A61B 2017/00672; A61B 2017/00637; A61B 17/0057

(57) ABSTRACT

A suturing device is provided that includes a suture positioning assembly that is slidably attachable to a sheath, a housing of the assembly includes a nose, a primary suture with connectors, an end effector, and an end effector actuator, an intravascular tip with a blood contact indicator on the nose, a slide limiter, and a fascia receiving surface, the intravascular tip is configured for insertion into the artery when disposed beneath a fascial layer, the fascia receiving surface is configured for accepting a perimeter edge of a dilated hole of the fascial layer, the housing nose further includes an intermediary suture having intermediary suture connectors, the primary suture connecters are configured for connection to the intermediary suture connectors, the end effector actuator is disposed to operate the end effector to condition the facial layer hole in a position for suturing by the intermediary suture connected to the primary suture.

### FIELD OF THE INVENTION

The invention relates generally to a suturing device. More specifically, the invention relates to a device and method for suturing a fascial connective tissue layer that is proximal to an region under treatment.

### BACKGROUND OF THE INVENTION

Many types of surgical procedures are being adapted from manual/open procedures to a minimally invasive endovascular approach. Surgical aortic valve replacement (SAVR) has transitioned in many high-risk and prohibitive-risk patients to trans-catheter aortic valve replacement (TAVR). Abdominal and thoracic aortic aneurysms are being treated through a percutaneous endovascular aneurysm repair (PEVAR). In order to perform many of these procedures, a large-bore sheath is used to access the vascular system of a patient through the femoral artery. This sheath is placed over a dilator and inserted through the wall of a blood vessel in order to obtain this access, and may thereafter be used for guiding medical instruments such as catheters, guide wires and the endovascular devices into the circulatory system of a living being.

To perform these procedures, access into the body through layers of tissue is required to reach the femoral artery. In gaining access to different portions of the body, different layers of tissue must be separated or penetrated. These layers can include skin, subcutaneous fat, fascia (e.g., connective tissue), muscle, arteries, veins, intestine, and other organs. Often, after access through these layers is achieved and the procedure is completed, these tissues must be approximated and sealed. The function of reapproximation can vary to include hemostasis, prevent hernia, contain gastrointestinal contents, etc.

Known in the art is the “Fascia Suture Technique” that uses a suture to reapproximate the fascial layers in the roof of the femoral triangle, which may include the fascia lata, cribriform fascia, and femoral sheath with the purpose of achieving hemostasis after arteriotomy is made in the femoral artery. This technique is employed by dissecting the dermal and adipose layers of tissue along the femoral access sheath, which remains in place following the procedure. Dissection is performed both proximal and distal to the femoral sheath until the fascia layers are exposed and visually identified. A suture is passed through the fascial layers surrounding the access site, and knotted in such a way as to create a purse-string closure surrounding the femoral access sheath. As the tapered sheath is withdrawn, the knot is tightened to achieve hemostasis by closing the fascia thus containing femoral blood loss to the space bounded by the tissues that define the femoral triangle (e.g. inguinal ligament, sartorius muscle, adductor longus muscle, cribriform fascia, fascia lata, psoas major muscle, iliacus muscle, and pectineus muscle) and by resisting systolic blood pressure within the enclosed space. The wire is left in place until hemostasis is confirmed, the wire is then removed and the knot is subsequently tightened and locked. What is needed is a fascia suture technique for enabling a percutaneous, device-based solution that precludes the requirement for manual dissection and visualization of the fascial layers during closure.

### SUMMARY OF THE INVENTION

To address the needs in the art, a suturing device is provided that includes a suture positioning assembly that is slidably attachable to an exterior of a sheath, where a housing of the suture positioning assembly includes a housing nose, a primary suture having primary suture connectors, an end effector, and an end effector actuator, where a distal end of the housing nose includes an intravascular tip, a slide limiter, and a fascia receiving surface, where the intravascular tip includes a blood contact indicator to receive blood flow from an artery, where the intravascular tip is configured for insertion into the artery when disposed beneath a fascial layer, where the fascia receiving surface is configured for accepting a perimeter edge of a dilated hole of the fascial layer, where the housing nose further includes an intermediary suture having intermediary suture connectors, where the primary suture connecters are configured for connection to the intermediary suture connectors, where the end effector actuator is disposed to operate the end effector to hold the facial layer hole in a position for suturing by the intermediary suture connected to the primary suture.

In one aspect of the invention, the end effector can include a straight needle, or a curved needle.

In another aspect of the invention, the housing nose further includes a tissue capturing device that is configured to deploy from the housing nose into, beneath, or above the fascial layer and draw the dilated hole in the fascial layer to the fascia receiving surface.

In a further aspect of the invention, the housing nose further includes a dilation controller, where the dilation controller is configured to change in size to alter a diameter of the dilated hole in the facial layer, where the dilation controller is configured to create resistance to the suture positioning assembly in being retracted from the facial layer along the lumen access sheath, where the created resistance is sufficient to indicate that the housing nose is securely held in place by circumferential tension of the dilated hole of the fascial layer on the fascia receiving surface. Here, the dilation control element can include an inflatable membrane, an expandable umbrella, or a shape changing collar.

In yet another aspect of the invention, the slide limiter is configured to induce an insertion resistance along the access sheath when the housing nose is desirably positioned on the facial layer, where the desirably positioned slide limiter establishes an upper bound against which the fascia will be stabilized and prevented from creeping further up the housing nose, where the desirably positioned slide limiter prevents the housing nose from passing through the fascia beyond the fascia receiving surface.

According to one aspect of the invention, the assembly slide limiter is a material that includes an elastomer, a complaint polymer, a complaint metallic element comprising nitinol, or an assembly of rigid elements designed to change shape upon contact with fascial layer.

In another aspect of the invention, the blood contact indicator includes a capillary, where the capillary spans from the housing nose to a proximal end of the housing, where a distal end of the capillary at the housing nose is configured to enter the body lumen, where blood flow through the capillary, or blood pressure in the capillary, or a presence of blood in the capillary is an indicator of the sheath being desirably position in an artery.

In a further aspect of the invention, the slidable attachment of the suture positioning assembly on the sheath includes clamping, hinged clamping, snap-fitting, stitching, or coaxial mating.

In on embodiment, the invention includes a suturing device that is slidably attachable to a sheath device, where the slidable attachment of the suturing device is configured to position a suture in an approximated state on a defect in a body lumen for suturing. Here, the body lumen can include an artery, a vein, or a bowel.

### BRIEF DESCRIPTION OF THE DRAWINGS

FIGS. 1A-1C show different views of a suture positioning assembly, according to embodiment of the current invention.

FIGS. 2A-2C show the intravascular tip inserted to an artery when disposed beneath a fascial layer, according to the current invention.

FIGS. 3A-3G show an exemplary detailed procedure of the device operation of the internal structures of the device, according to the current invention.

FIGS. 4A-4C show embodiments of the shapes of example end effector shapes, according to the current invention.

FIGS. 5A-5C show different views of an alternate suture positioning assembly, according to embodiment of the current invention.

FIGS. 6A-6E show alternative mating mechanisms of the suture positioning assembly with the sheath, according to the current invention.

FIGS. 7A-7C show different embodiments of the housing nose having a dilation controller that is configured to change in size to alter a diameter of said dilated hole in facial layer, according to the current invention.

FIG. 8 shows a flow diagram of the implementation and use of the current invention.

### DETAILED DESCRIPTION

The current invention provides a surgical procedure and closure device that approximates and closes a defect in a tissue layer, such as a tissue layer superior to a blood vessel. In one example, the tissue defect may arise from the external introduction of an instrument for vascular or otherwise internal access to the body, e.g. percutaneous or surgical intervention. The tissue layer may be the femoral sheath, fascia lata and/or cribriform fascia that covers the roof of the femoral triangle, or other connective tissue layer e.g. abdominal fascia. The closure of such a tissue may allow for hemostasis to occur in the enclosed femoral triangle in order to create a natural method of tamponade around the femoral artery after removal of the procedural sheath. The ability to exert a greater compressive force around the arteriotomy from closure of the fascia above and the surrounding borders of the femoral triangle may allow the arteriotomy to naturally resolve. The anatomy of the femoral triangle includes the floor made up of the following muscles from lateral to medial: iliacus, psoas major, pectineus, and adductor longus; the superior border is made up of the inguinal ligament; the medial border is the adductor longus muscle; and the lateral border is comprised of the sartorius muscle. The current invention eliminates procedural steps that include opening the skin, manually dissecting down to the fascia, and directly suturing the fascia to close these internal tissues.

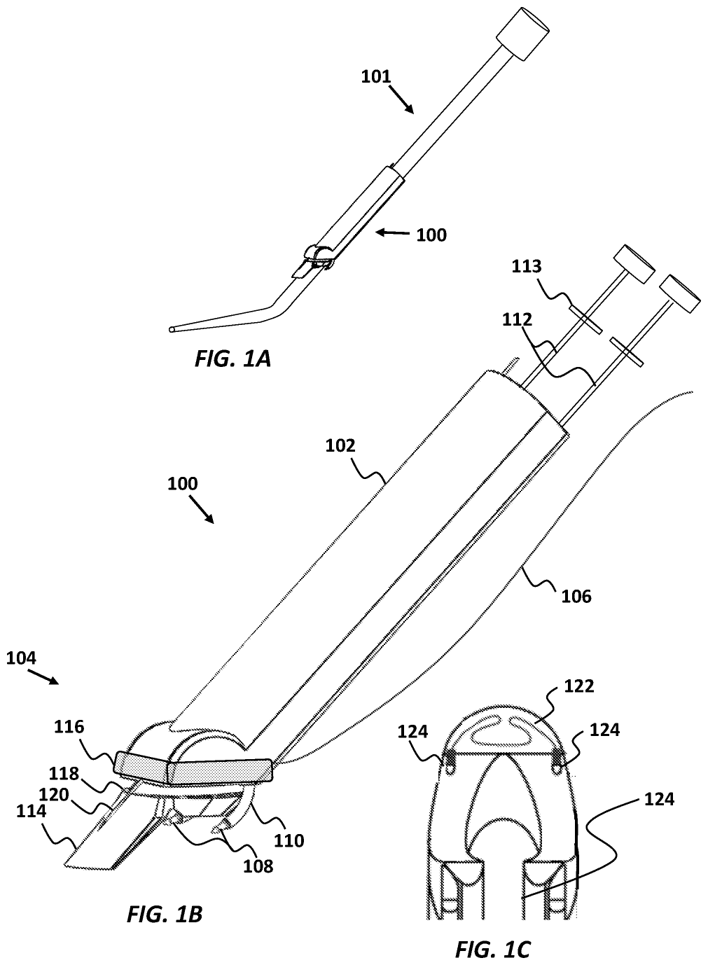

Turning now to the drawings, FIGS. 1A-1C show different views of a suture positioning assembly 100 that is slidably attachable to an exterior of a sheath 101. As shown in FIGS. 1B-1C, the housing 102 of the suture positioning assembly 100 includes a housing nose 104, a primary suture 106 having primary suture connectors 108, end effector 110 that can be independently deployed, and an end effector actuator 112, wherein a distal end of said housing nose comprises an intravascular tip 114, a slide limiter 116, and a fascia receiving surface 118, where the intravascular tip 114 comprises a capillary tube 120 to receive blood flow from an artery.

In one aspect of the invention, the assembly slide limiter includes a material that includes a compliant elastomer that, upon contact with the proximal surface of the fascia, will deform and/or buckle, adding additional cross-sectional area to the upper bounding surface of the slide limiter, creating a broader upper bound of the slide limiter with which to prevent the fascia from creeping up further up the housing nose beyond the fascia receiving surface. Here, the slide limiter can be the elastomer, complaint polymer, complaint metallic element comprising nitinol, or an assembly of rigid elements designed to change shape upon contact with fascial layer.

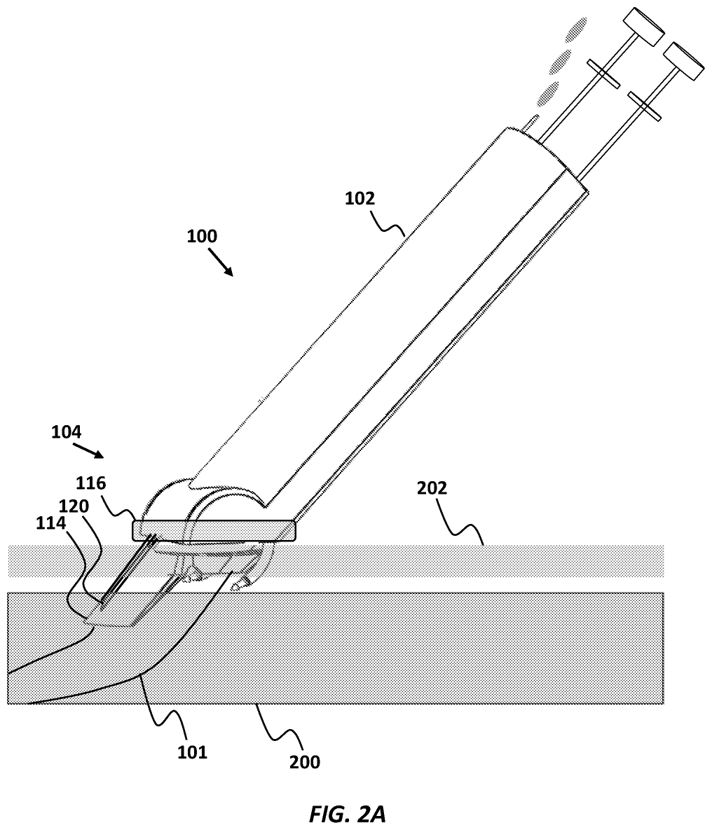

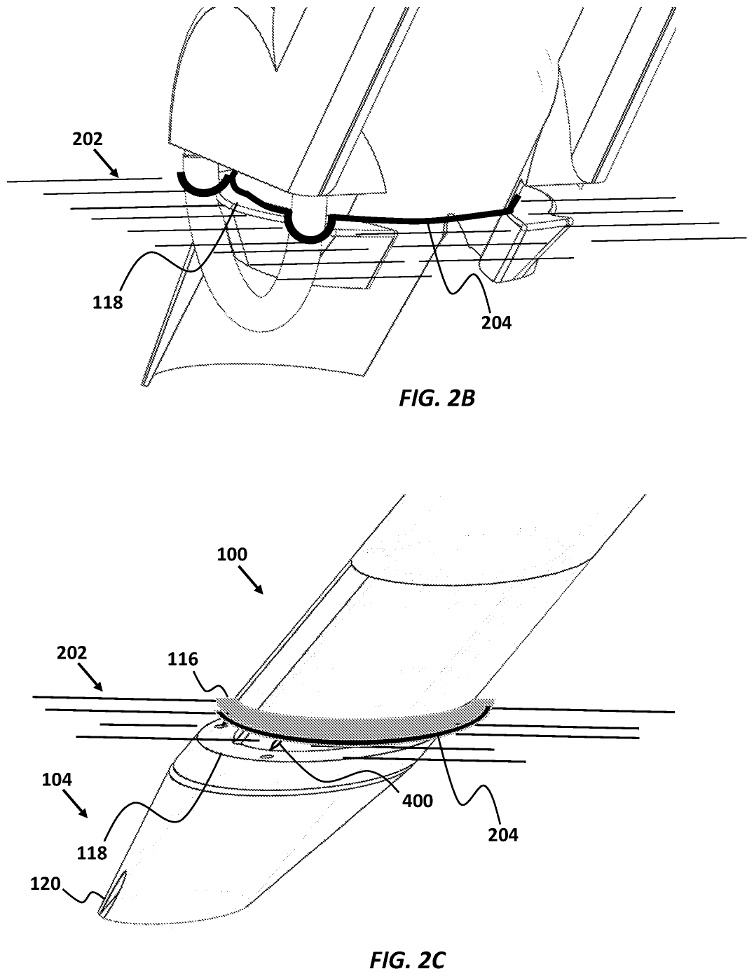

FIG. 2A, shows the intravascular tip 114 inserted to artery 200 when disposed beneath a fascial layer 202, where the fascia receiving surface 118 (for example having a ledge-shape) is configured for accepting a perimeter edge of a dilated hole (defect) 204 of the fascial layer as shown in FIGS. 2B-2C, where in FIG. 2B the slide limiter 116 is not shown for clarity. Further shown in FIG. 2C is an alternate embodiment of the suture positioning assembly 100 that has a conical housing nose 104, and linear elements for the end effectors 400 and suture assemblies described below.

FIG. 1C shows the housing nose 104 further including an intermediary suture 122 having intermediary suture connectors 124, where the primary suture connecters 108 are configured for connection to the intermediary suture connectors 124. According to the invention, the end effector actuator 112 is disposed to operate the end effector 110 to hold the facial layer hole in a position for suturing by the intermediary suture connected 112 to said primary suture 106.

The current invention enables one to approximate tissues during or following interventional medical procedures, such as open surgery, laparoscopic surgery, endoscopy, endovascular surgery and procedures, and cardiac catheterizations is portrayed. The invention may be used in a percutaneous or open surgical fashion and approximate a variety of tissues, such as skin, subcutaneous fat, fascia, muscle, or vascular tissue. Further, the tissue approximation device may be used with the intent of sealing tissue to achieve vascular hemostasis, prevent hernias, obtain watertight or airtight closure of tissue layers, or approximate and close disjointed tissues, for example. Tissue sealing of any of the previously described tissues may be achieved by a variety of mechanical techniques such as suture-like materials, clips, hook-and-loop fasteners, patches, plugs, clamps, and/or biological and synthetic adhesives. The invention may be placed directly on the tissue of interest or be integrated with an instrument of preference, such as a cardiovascular sheath, a guidewire for maintaining access to a vessel or procedural space, or a laparoscopic surgery port, trocar, or other surgical or procedural access instrument.

FIG. 1A shows an example interface of the suture positioning assembly 100 as coaxially mated with a tubular access instrument 101, in this case, a cardiovascular access sheath. The cardiovascular sheath 101 serves as a guide along which the device 100 travels during operation. The coaxial mate enables the device 100 to follow the path of the access instrument 101 through the skin, and any subcutaneous adipose or loose connective tissues, in order to position itself against an access-induced defect in a known anatomical structure, e.g. the cribriform fascia, fascia lata, or femoral sheath. The device 100 may be used to manipulate a known anatomical structure in order to approximate and close an access-induced defect in the structure. The device is inserted, manipulated, and subsequently removed to induce the desired tissue closure effect, leaving behind only the mechanical components necessary to maintain tissue closure.

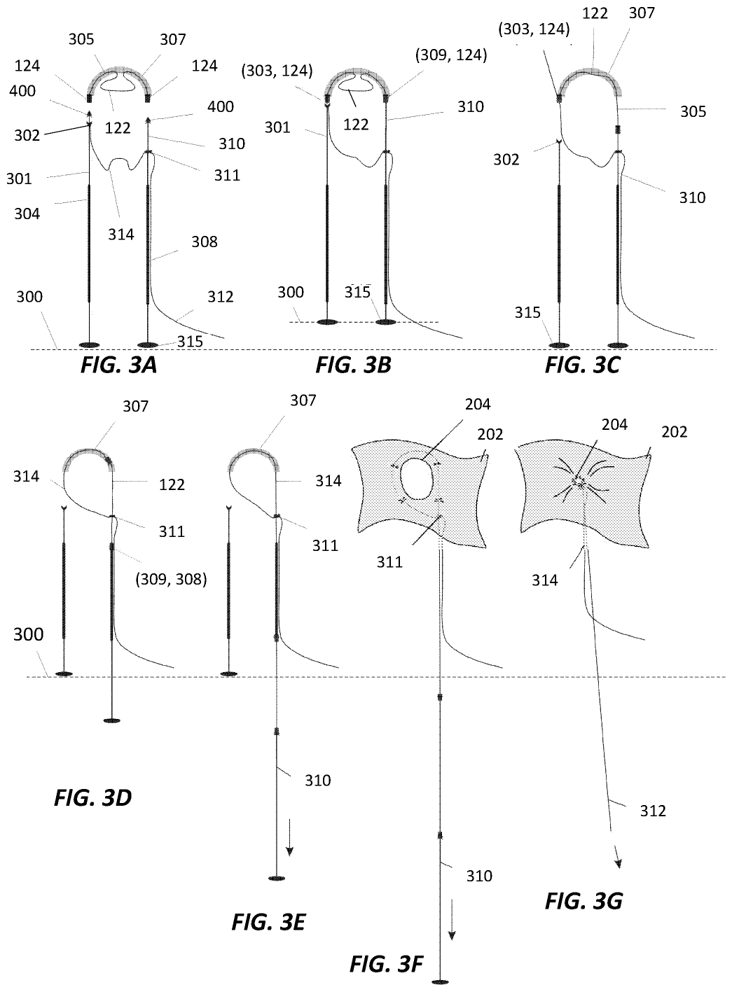

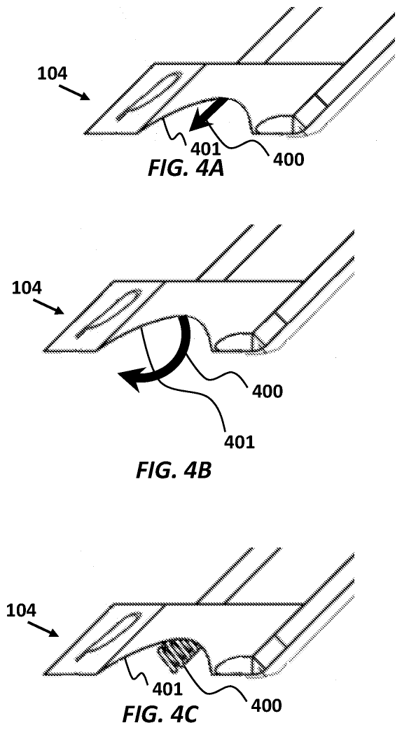

FIGS. 3A-3G show an exemplary detailed procedure of the device operation of the internal structures of the device 100 and their designated operations to deliver suture-mediated closure to a tissue defect in a subcutaneous tissue layer of interest. In this example, once the user has passed the streamlined or tapered device nose 104 around the access instrument 101 and through the superficial tissue layers to reach the level of the subcutaneous tissue of interest 200, the user may deploy end effectors 400 (see FIGS. 4A-4C) to capture the tissue of interest 202 (e.g., fascia) and the retract the tissue of interest 202 into the receiving feature of the device nose 104 effecting the desired fold or “tent” in the tissue of interest. The device is now primed for suture deployment. FIGS. 3A-3G show a schematic views of the internal structures and mechanisms of the device 100 as stored for transportation and handling. Reference position 300 indicates the stored position of end effectors 400. End effector 400 may be a barbed suture needle that is mechanically mated with its axial shaft 301 via a press-fit or similarly tenuous junction 302 capable of releasing end effector 400 from its axial shaft 301 upon the application of tension to the three-part system that resides and travels within device body lumen 200. One end 314 of a suture system (314, 311, 312), which in combination make up the primary suture 106, may be permanently affixed to end effector 303 such that any movement of end effector 303 is followed by its attached suture end 314. As shown previously, FIG. 3A details intermediary suture 122 as attached on both ends to suture cuffs 124. The stored portion of intermediary suture 122 may reside in the nose 104 of the device body 100, where upon the application of tension to one end of the intermediary suture 122, the entirety of intermediary suture 122 will travel within and follow the curved path of an interior feature 307 of device nose 104. The interior feature 307 may be a shelf or channel that provides a radial force to the intermediary suture and subsequent flexible sutures as they are drawn through the feature around the cylindrical access instrument, so as to prevent contact of the suture with the device body. The interior feature 307 may not restrict any suture from dropping out of the “active surface” of device body 100.

End effector 400, which may be a barbed needle tip, is permanently affixed to its axial shaft 310 housed within device body lumen 304. The axial shaft 310 of the end effector 400, as described previously, is stored such that it passes through a pre-tied surgical knot 311 comprised of the junction between a suture end 314 that is stored internal to the device within device lumen 304 and an opposing suture end 312 that may be stored external to the device or within lumen 304.

When tissue has been folded or tented into the receiving surface, for example a concave feature or planar shelf, of device nose 104 via deployment and retraction of end effectors 400, the user may actuate end effectors 400 by applying compressive force to their axial shafts 301 and 310 via handles or actuators 315 to pass the end effectors through the fold in the tissue of interest, as shown in FIG. 3B. The passing of end effectors 400 creates two straight passages through folded tissue on either side of the access instrument e.g. cardiovascular sheath for a total of four holes radially arranged around the defect in the tissue of interest. As such, the end effectors enter the tissue from the side of the tissue layer that is proximal to the device user, puncture into and return out of the tissue on the side of the tissue layer that is proximal to the device user. The actuators of axial shafts 301 and 310 may now reside in reference position 300 as shown in FIG. 3B. End effector 400 may have now formed a mechanical mate with suture cuff 124 forming barb/cuff complex (400, 124), and the opposing effector 400 may have likewise mated with suture cuff 309. Stored suture end 314 may have been partially drawn out from lumen 307.

Shown in FIG. 3C, axial shafts 301 and 310 may be retracted from the tissue. Due to the suture/cuff complexes 303, 124, the axial shaft 301 and tenuous mechanical junction (e.g. socket, or cradle) 302 may be allowed to travel back through the tissue having detached from end effector 400. Actuator 315 of axial shaft 301 may return to reference position 300 and remain there. Axial shaft 310 is now mechanically coupled to barb/suture cuff complex 309, 124, stored and resident intermediary suture 305, 122, barb/suture cuff complex 303, 124 and previously stored suture end 314. This 6-part system may now be drawn in tension, as shown in FIG. 3D, by actuator handle 315 of axial shaft 310 through the pre-tied suture knot 311, which remains housed in the distal end of the axial shaft port. Actuator handle 315 may now surpass reference position 300 as the 6 part system is drawn through the tissue, and axial shaft 310 is drawn away from the device, towards the device user. By drawing this 6 part system through the structural guide channel 307 in the nose 104 of the device 100, the intermediary suture and cuff complexes draw the suture end 314 through pre-tied surgical knot 311, the remaining end of which belongs to the same filament of suture 312. The tissue defect is now surgically tied, and while, in the event of a cardiovascular access closure, the guiding wire must remain intraarterial, the access instrument may be removed by the user while the user applies tension to suture end 312, the suture “rail”, and pushes suture knot 311 towards the defect 204 in the tissue of interest 202, as shown in FIG. 3F. The intermediary suture 112 and cuff complexes may now be trimmed from the single knotted suture, and the knot may be further pushed either manually or via a user-activated tensioning mechanism within the device so as to create a purse-string closure of defect 204 using a U-stitch or analogous surgical closure pattern in the tissue of interest 202 as shown in FIG. 3G. In the case of a cardiovascular access closure, the guidewire is still in place. As the user determines the closure to be effective, the user or device may continue to apply tension to suture “rail” 312, and compressive force to knot 311 as the user removes the guiding wire and completes the knotted, purse-string closure of the tissue of interest. For the indication of achieving hemostasis after closure of the tissue of interest, the device may embed indicators in the housing, such as a cannula, needle or wick to inform the user if hemostasis was or was not achieved after securing the approximated tissue.

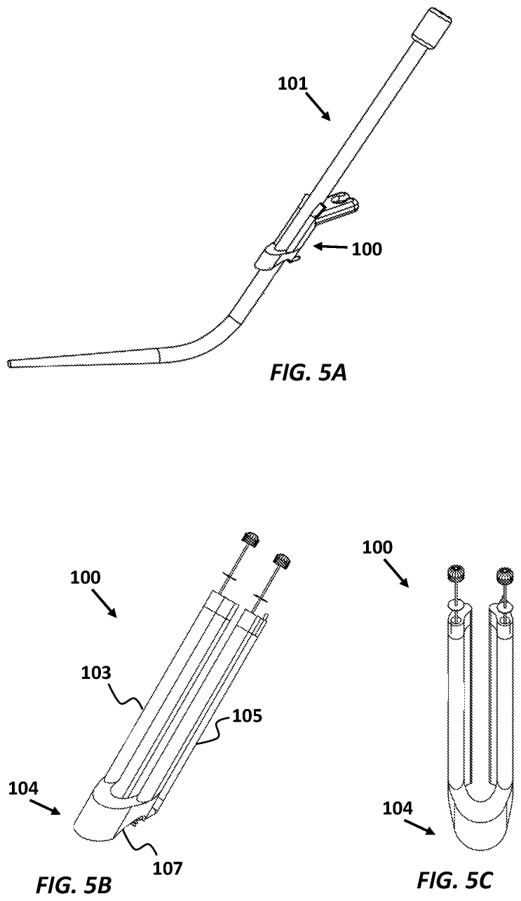

To achieve a coaxial mate with the suture positioning assembly 100 as shown in FIG. 1A, the tissue approximation device may be snapped, pressed, or otherwise mechanically secured onto the instrument, e.g. a cardiovascular access sheath 101 via an axial slit 124 (see FIG. 1C, FIGS. 5A-5C). The axial slit 124 and corresponding structural boundaries may be, but are not limited to a blunt, sharp, circular, convex, or cone shape to allow tissue, such as subcutaneous fat, to slide over the tissue approximation device and mitigate damage, entanglement or interference with the tissue before reaching the desired tissue layer and tissue defect to be approximated, which may include a subcutaneous tissue layer such as the fascia or arterial wall.

In a further aspect of the invention, the slidable attachment of the suture positioning assembly on the sheath to achieve coaxial mating of the suture positioning assembly with the sheath includes clamping, hinged clamping, clamping via hinged cams, springed hinges, snap-fitting, stitching, tongue-and-groove mating, dove-tail mating, ratchet mating, threaded connnectors or coaxial mating. The suture positioning assembly may be a single deformable component so as to achieve the coaxial mate with the sheath, or it may be comprised of discrete halves to be radially/circumferentially oriented around the sheath so as to mate with each other and create a coaxial mate, or a mate with which the central axes of the suture positioning assembly and of the sheath are offset but remain parallel.

Shown in FIGS. 5A-5C is one possible embodiment of a suture positioning assembly 100 to achieve vascular hemostasis. FIG. 5A shows one embodiment of the invention, where a device is provided as an integrated sheath and closure device. In further embodiments, the device may be equipped with a streamlined or tapered introductory access nose 104 intended to dilate any tissue (e.g. skin and adipose tissues) that surrounds the access sheath while traveling along the sheath towards the tissue defect of interest. To appropriately approximate to and manipulate the tissue of interest, the tissue-facing surface, or “active surface” of the device nose 104 may be angled relative to the axis of the device body 100 so as to achieve a parallel approximation with the tissue layer of interest. The “active surface” of the device may thus be oriented relative to the anterior longitudinal surface of the device body 100 at an angle of between 14 and 61 degrees. The active surface of the device may also follow convex contours designed to mate seamlessly with the tissue layer of interest, around the defect of interest. The device is equipped with two symmetrical, axial lumens 103 through which tissue end-effectors may pass and be articulated, directed, or manipulated. The device is equipped with two additional symmetrical lumens 105 that likewise house tissue end effectors that may be passed, articulated, directed or manipulated to puncture the tissue of interest and deploy mechanical closure components, e.g. sutures. Lumens 105 may contain baffles interior to the lumen in order to guide any flexible internal tissue end effectors in the appropriate direction. The device is equipped with a concave feature 107 within the nose 104 that will serve as a contour along which to shape the tissue and tissue defect, as well as providing a dedicated volume of space within which the tissue may be manipulated during the closure operation. In no way is the dedicated volume created by concave feature 107 restrictive of the area in which end effectors may pass or within which tissue may be manipulated.

The material of the device, particularly in, but not limited to the location of the nose 104 may be sufficiently flexible so as to allow the elastic deformation of the nose or device body and subsequent coaxial mating of the suture positioning assembly 100 with the access sheath 101. The mating surfaces of the suture positioning assembly 100 with sheath 101 may incorporate gaskets, bushings, springs, hydrophilic coatings or other mechanism(s) to ensure a tight, coaxial sliding junction with the access instrument. The independence of the suture positioning assembly 100 from the sheath 101 enables the device to perform the intended tissue manipulation either at the start of a procedure or at the end of a procedure, without the mandate that the access sheath be removed and “threaded” through the device in order to obtain a coaxial orientation of the two components.

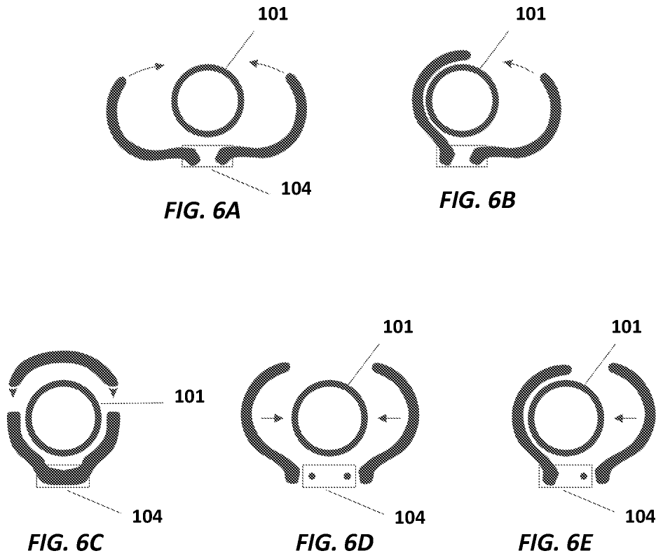

FIGS. 6A-6E show alternative mating mechanisms of the suture positioning assembly 100 with sheath 101, e.g. cardiovascular sheath. The device 100 may coaxially mate to a cylindrical instrument 101 via a single hinge (FIG. 6A) or dual-hinge (FIG. 6B) clamshell-type approximation mechanism, such that one or both halves of the end effector of the device may swing open, allowing the device to be placed around the sheath and subsequently closed and secured. Rather than an axial slit or clamshell, the device may also occupy a modular form factor as depicted in FIG. 6C. In one embodiment, the nose 104 or the body of the device 100 to be mated with an access instrument may be removed or disconnected from the device (FIG. 6D) to allow coaxial mating with the sheath, and subsequently replaced to form a circumferential sliding junction with the access instrument. The device body may also be split on the transverse plane as in FIG. 6D such that one half of the device is bound to the other around the access instrument, or such that each half is bound to a core body member. FIG. 6E depicts one half of the device bound to a core body member; the remaining modular half may be joined to the core body member around the access instrument.

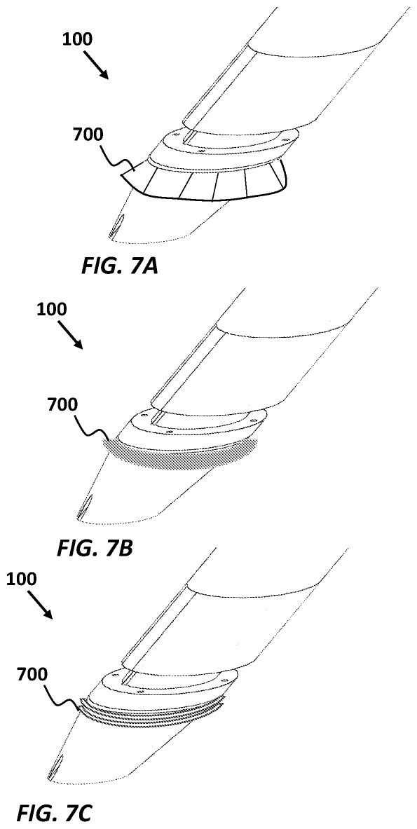

According to further embodiments of the invention, the housing nose 104 can also include a dilation controller, where the dilation controller is configured to change in size to alter a diameter of said dilated hole in facial layer 202, where the dilation controller is configured to create resistance when the suture positioning assembly 100 is retracted from the facial layer 201 along said lumen access sheath 101, where the created resistance is sufficient to indicate that the housing nose 104 is securely held in place by circumferential tension of the dilated hole 204 of said fascial layer on said fascia receiving surface. FIGS. 7A-7C show different embodiments dilation control element 700 that can include an expandable umbrella (see FIG. 7A), an inflatable membrane (see FIG. 7B), and a shape changing collar (see FIG. 7C). Here, the dilation control element can be deformable underneath the fasica layer so as to form an “anchor” or “T-tag”-style expansion device so as to prevent the user from pulling the device out of position while operating. In one embodiment, this feature could then be relaxed to reduce its cross section to original size and allow the device to be removed/slid back along the sheath towards the user thus disengaging from the fascia.

In one embodiment, the end effectors may be a coiled wire with sufficient stiffness to puncture and secure into a biological tissue of interest for subsequent pushing, pulling, and analogous tissue manipulation. The end effectors may embody or utilize similar tissue securement mechanisms such as opposing graspers, hooks, barbs, suction, or adhesives. After approximation of the suture positioning assembly 100 to the tissue of interest 202, the end effectors 400 may be secured to the tissue of interest by rotating and applying axial compression to their axial shafts via handles/actuators at the end of the end-effector axial shaft proximal to the user, in the direction of the tissue of interest, which effectively captures the tissue of interest e.g. fascia. The travel of the end effectors is limited by a mechanical hard stop, or “shoulder” feature 113 (see FIG. 1B) on the axial shaft of the end effectors. Once in contact with the device body 100, the “shoulder” 113 will stop travel of the end effectors so as to avoid damage of delicate tissues or structures beneath the tissue of interest, e.g. fascia. The tissue of interest may then be retracted into the interface surface, such as the concave feature 401 of FIGS. 4A-4C of the device nose 104 by applying tension to shaft of the end effectors (not shown0, creating a folding or “tenting” effect of the tissue of interest within the nose 104 of the device body 100.

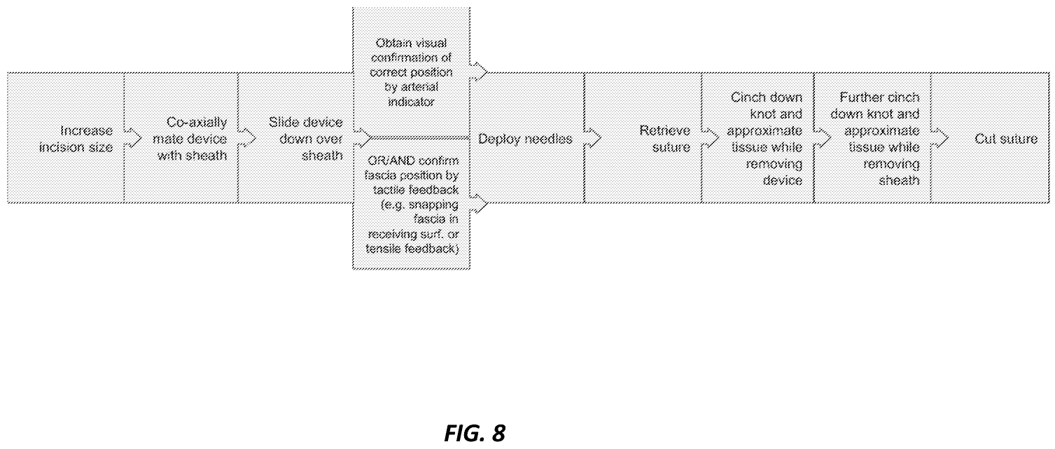

FIG. 8 shows a flow diagram of the implementation and use of the current invention that includes increasing the incision size, co-axially mating the device with a sheath, sliding the device down over the sheath, then obtaining visual confirmation of the correct positioning of the device by an arterial indicator, and/or confirming the fascia positioning by use of a tactile feedback, such as snapping fascia in receiving surface or a tensile feedback. The next steps include deploying the needles, retrieving the suture, cinching down the knot and approximating the tissue while removing the suturing device, then further cinching down the knot and approximating the tissue while removing the sheath, and finally cutting the suture.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example the needles themselves could be curved or straight, flexible or rigid, etc while still performing the mating of “barb” with “cuff” by traversing the tissue layer once, twice, or up to four times per needle.

All such variations are considered to be within the scope and spirit of the present invention as defined by the following claims and their legal equivalents.

What is claimed:

1) A suturing device, comprising a suture positioning assembly that is slidably attachable to an exterior of a sheath, wherein a housing of said suture positioning assembly comprises a housing nose, a primary suture having primary suture connectors, an end effector, and an end effector actuator, wherein a distal end of said housing nose comprises an intravascular tip, a slide limiter, and a fascia receiving surface, wherein said intravascular tip comprises a blood contact indicator to receive blood flow from an artery, wherein said intravascular tip is configured for insertion into said artery when disposed beneath a fascial layer, wherein said fascia receiving surface is configured for accepting a perimeter edge of a dilated hole of said fascial layer, wherein said housing nose further comprises an intermediary suture having intermediary suture connectors, wherein said primary suture connecters are configured for connection to said intermediary suture connectors, wherein said end effector actuator is disposed to operate said end effector to hold said facial layer hole in a position for suturing by said intermediary suture connected to said primary suture.

2) The fascia suturing device of claim 1, wherein said end effector is selected from the group consisting of a straight needle, and a curved needle.

3) The fascia suturing device of claim 1, wherein said housing nose further comprises a tissue capturing device that is configured to deploy from said housing nose into, beneath, or above said fascial layer and draw said dilated hole in said fascial layer to said fascia receiving surface.

4) The fascia suturing device of claim 1, wherein said housing nose further comprises a dilation controller, wherein said dilation controller is configured to change in size to alter a diameter of said dilated hole in said facial layer, wherein said dilation controller is configured to create resistance to said suture positioning assembly in being retracted from said facial layer along said lumen access sheath, wherein said created resistance is sufficient to indicate that said housing nose is securely held in place by circumferential tension of said dilated hole of said fascial layer on said fascia receiving surface.

5) The fascia suturing device of claim 5, wherein said dilation control element is selected from the group consisting of an inflatable membrane, an expandable umbrella, and a shape changing collar.

6) The fascia suturing device of claim 1, wherein said slide limiter is configured to induce an insertion resistance along said access sheath when said housing nose is desirably positioned on said facial layer, wherein said desirably positioned slide limiter establishes an upper bound against which said fascia will be stabilized and prevented from creeping further up said housing nose, wherein said desirably positioned slide limiter prevents said housing nose from passing through said fascia beyond said fascia receiving surface.

7) The fascia suturing device of claim 1, wherein said assembly slide limiter comprises a material selected from the group consisting of elastomer, complaint polymer, complaint metallic element comprising nitinol, or an assembly of rigid elements designed to change shape upon contact with fascial layer.

8) The fascia suturing device of claim 1, wherein said blood contact indicator comprises a capillary, wherein said capillary spans from said housing nose to a proximal end of said housing, wherein a distal end of said capillary at said housing nose is configured to enter said body lumen, wherein blood flow through said capillary, or blood pressure in said capillary, or a presence of blood in said capillary is an indicator of said sheath being desirably position in an artery.

9) The fascia suturing device of claim 1, wherein said slidable attachment of said suture positioning assembly on said sheath comprises clamping, hinged clamping, snap-fitting, stitching, or coaxial mating.

10) A suturing device, wherein said suturing device is slidably attachable to a sheath device, wherein said slidable attachment of said suturing device is configured to position a suture in an approximated state on a defect in a body lumen for suturing.

11) The suturing device of claim 10, wherein said body lumen is selected from the group consisting of an artery, a vein, and a bowel.
